(19) United States
(12) Patent Application Publication
(10) Pub. No.: US 2003/0109486 A1
(43) Pub. Date: Jun. 12, 2003

(54) Methods and therapeutic compositions for utilization of adenosine 5'-triphosphate (ATP) in the treatment of acute lung injury (ALI) and acute respiratory distress syndrome (ARDS)

(76) Inventor: Eliezer Rapaport, Belmont, MA (US)

Correspondence Address: CONNOLLY BOVE LODGE & HUTZ LLP, SUITE 800, 1990 M STREET NW, WASHINGTON, DC, 20036-3425, US

(21) Appl. No.: 10/284,096

(22) Filed: Oct. 31, 2002

Related U.S. Application Data
(60) Provisional application No. 60/330,827, filed on Oct. 31, 2001.

(51) Int. Cl. A61K 31/7076; A61L 9/04

(52) U.S. Cl. 514/47; 424/45

(57) ABSTRACT

The invention discloses methods for the utilization of adenosine 5′-triphosphate (ATP) in the treatment of acute lung injury (ALI) and acute respiratory distress syndrome (ARDS). The administration of ATP results in several therapeutic activities, which by acting in consort provide the methods for treatment of ALI and ARDS. These therapeutic activities are 1. The utility of ATP as a preferential pulmonary vasodilator, 2. The utility of the catabolic product of ATP, adenosine, as an anti-inflammatory agent, 3. The anti-thrombotic, pro-fibrinolytic activities of ATP and adenosine, and 4. The utility of ATP in improving organ and muscle function in advanced disease patients. Therapeutic compositions and procedures for treating ALI and ARDS by administration of ATP are provided.

### TECHNICAL FIELD

The present invention relates to treating acute lung injury (ALI) and Acute Respiratory Distress Syndrome (ARDS). In particular, according to the present invention a mixture of adenosine and an inorganic phosphate and/or an adenine nucleotide and especially ATP is administered to a patient suffering from ALI or ARDS.

### BACKGROUND ART

Acute lung injury (ALI) and acute respiratory distress syndrome (ARDS) are considered to be a consequence of an aggressive inflammatory response accompanied by pulmonary hypertension, which can lead to multiple organ failure and a high rate of mortality ranging from 35% to 50%. The inflammatory response is the result of inflammatory cell migration into interstitial and alveolar spaces followed by release of proteases and reactive oxygen intermediates. Pulmonary hypertension is the clinical outcome of activation and release of constrictive vasoactive mediators, which is accompanied by diffuse pulmonary microvascular thrombosis (Kollef et al., The acute respiratory distress syndrome. New Engl J Med 1995; 332:27-37, 1995; Ware et al., The acute respiratory distress syndrome. New Engl J Med 2000; 342:1334-1349, 2000).

Sepsis, severe trauma, aspiration of gastric content, adverse outcome of cardiothoracic surgery or massive blood transfusion are the most common clinical events that place patients at risk for developing ALI and ARDS. Supportive care of mostly mechanical ventilation constitutes the current state of the art treatment for ALI and ARDS patients in a critical care environment (Tobin, Culmination of an era in research on the acute respiratory distress syndrome. New Engl J Med 2000; 342:21360-1361, 2000). It is widely acknowledged that no pharmacologic therapies have yet proved efficacious in the treatment of ALI and ARDS (The ARDS Network Authors, Ketoconazole for early treatment of acute lung injury and acute respiratory distress syndrome. A randomized controlled trial. JAMA 2000; 283:1995-2002).

U.S. Pat. No. 6,001,842 suggests methods for preventing or treating endotoxin-related tissue injury by administration of a P2X purinoceptor antagonist. ALI and ARDS are the commonest adverse clinical outcome of endotoxin-related lung injury. The P2X purinoceptors are a class of ATP receptors (see Section 2, Description of Prior Art). U.S. Pat. No. 6,001,842 teaches that synthetic antagonists of ATP, which is the natural, endogenous ligand for the P2X purinoceptors, are effective in preventing and treating sepsis (endotoxin-related)-produced acute lung injury. Thus, U.S. Pat. No. 6,001,842 in a most direct fashion, teaches away from the present invention by disclosing the utilization of an antagonist of ATP for-the treatment of the same clinical indication.

A variety of pharmacological agents have been unsuccessfully utilized in attempted therapeutics interventions in ALI and ARDS. These included pulmonary vasodilators, anti-inflammatory agents, anti-oxidants, inhibitors of thromboxane synthetase, exogenous surfactants, inhaled vasodilators (nitric oxide), anti-endotoxin and anti-cytokine agents (Kollef et al., supra and Ware et al et al., supra). ALI and ARDS, being a form of noncardiogenic pulmonary edema resulting from acute damage to the alveoli, need the time bought by pharmacologic intervention in order for the lungs to heal. The present disclosure of continuous superior vena cava administration of ATP in the treatment of ALI and ARDS teaches how to capitalize on the first passage effects of ATP and the properties of its catabolic product generated in situ, adenosine.

The roles of intracellular adenosine 5′-triphosphate (ATP) as the major cellular energy source, a phosphate and adenylyl groups donor, an intermediate in numerous biosynthetic reactions and an allosteric regulator of a variety of cellular proteins, have been well-established. Only in the past fifteen years have the roles of ATP and its major catabolic product, adenosine, began to emerge as powerful physiological, extracellular regulators of intravascular, extravascular and central nervous system functions. These roles are attracting significant attention within the field of drug development. Extracellular ATP has been shown to regulate cardiac function, vascular tone, muscle function, neurotransmission and liver metabolism. Extracellular ATP is produced by specific release of ATP from cells and it interacts with families of purinergic receptors present on the plasma membrane of virtually every cell type. Extracellular ATP is the source of extracellular adenosine, which interacts with its own family of receptors. These adenosine and ATP receptors are of two major classes, the P1 and P2 receptors. The adenosine receptors (P1) are subdivided into A1, A2alpha, A2beta and A3, which in turn activate phospholipase C (A1, A2beta, A3), regulate adenylate cyclase function (A2alpha, A2beta) and modulate ion channels activities (A1, A2beta, A3). The ATP receptors (P2) belong to a subgroup that acts via G-protein coupled receptors (P2Y) and a subgroup of intrinsic ion channels (P2X). Activation or antagonism of specific receptors, produce immediate and diverse biological responses (Reviewed by Agteresch et al., Adenosine Triphosphate. Established and potential clinical applications. Drugs 1999; 58:211-232,1999). The utilization of exogenous ATP administration for the treatment of a variety of clinical indications is based on its ability to expand total blood (red blood cell), blood plasma (extracellular) and organ pools (steady state levels) of ATP (Rapaport et al., Anticancer activities of adenine nucleotides in mice are mediated through expansion of erythrocyte ATP pools. Proc Natl Acad Sci USA 1989a; 86(5):1662-6 and Rapaport et al., Generation of extracellular ATP in blood and its mediated inhibition of host weight loss in tumor-bearing mice. Biochem Pharmacol 1989b; 38(23):4261-6). The ability of intravenously administered ATP to immediately expand total blood ATP pools and blood plasma ATP levels has now been confirmed by pharmacokinetic studies, which were part of several human clinical trials (Haskell et al., Phase I trial of extracellular adenosine 5′-triphosphate in patients with advanced cancer. Medicinal and Pediatric Oncology 1996; 27(3):165-173; Agteresch et al., Randomized Clinical Trial of adenosine 5′-triphosphate in patients with advanced non-small-cell lung cancer. J Natl Cancer Inst 2000; 92(4):321-8; Agteresch et al., Pharmacokinetics of intravenous ATP in cancer patients. Eur. J. Clin. Pharmacol. 2000; 56:49-55).

Administration of adenine nucleotides (ATP, ADP, AMP or any other adenine nucleotide) into the systemic circulation or into extravascular sites, results in the rapid degradation of the nucleotides to adenosine and inorganic phosphate due to the presence of strong ecto-enzymatic (on the plasma membrane of cells) and soluble catabolic enzymatic activities (Rapaport et al., supra; Agteresch et al., supra). These catabolic activities are followed by the immediate incorporation of adenosine and inorganic phosphate into liver ATP pools, yielding expansions of these ATP pools. Detailed experimental animal studies have demonstrated that the turnover of the expanded liver ATP pools supply the adenosine precursor in the hepatic sinusoids, for the increased salvage synthesis of red blood cell ATP pools (Rapaport et al., supra. and Rapaport et al., supra).

Red blood cell utilize only salvage precursors (mostly adenosine) for ATP synthesis by glycolytic pathway only and do not engage in either de novo synthesis of ATP or in oxidative phosphorylation. Thus, the exogenously administered ATP produces elevated liver ATP pools, which in turn yield elevated red blood cell ATP pools. Subsequently, the expanded red blood cell ATP pools are slowly released into the blood plasma (extracellular) by a non-hemolytic mechanism. The continuous release of micromolar amounts of ATP from red blood cells results in elevated pools of extracellular ATP in the blood, in spite of the intravascular presence of strong catabolic activities. The elevated levels of ATP and its catabolic product, adenosine, can then affect a multitude of physiological functions by activating P2 (ATP) and P1 (adenosine) receptors. As importantly, the release of red blood cell ATP followed by its degradation to adenosine, act not only in the regulation of vascular tone, especially in the pulmonary circulation (Sprague et al., ATP: the red blood cell link to NO and local control of the pulmonary circulation. Am J Physiol 1996; 271:H2717-H2722, 1996), but also supplies purine precursors for salvage synthesis of ATP in peripheral sites (Dietrich et al., Red blood cell regulation of vascular tone through adenosine triphosphate. Am J Physiol 2000; 278:H1294-H1298).

It is important to note that purine (adenosine and ATP) losses, affecting organ and muscle function, were reported both in advanced diseases (Scheenberger et al., Effect of cancer on the in vivo energy state of rat liver and skeletal muscle. Cancer Res 1989; 49:1160-1164; Shen et al., 1999; Agteresch et al., supra) and upon aging (Rabini et al., Diabetes mellitus and subjects' ageing: a study on the ATP content and ATP-related enzyme activities in human erythrocytes. Eur. J. Clin. Invest. 1997; 27:327-332) in experimental animals and humans respectively.

TABLE 1
Effects of Adenosine 5′-Triphosphate (ATP) and Adenosine with Potential
or Demonstrated Clinical Implications (reproduced from Agteresch et al.,
1999, supra)

| Topic | Cells/Organ | Animals | Patients |
|---|---|---|---|
| Anesthesia and analgesia | | | |
| Blood pressure during | | | Reduced |
| surgery | | | |
| Anaesthetic requirement | | Reduced | Reduced |
| Opioid requirement after | | | Reduced |
| surgery | | | |
| Pain | | Reduced | Reduced |
| Pulmonary hypertension | | Reduced | Reduced |
| Supraventricular tachycardias | | | Inhibited |
| SA + AV node conduction | Inhibited | | Inhibited |
| Mechanism wide QRS complex | | | Diagnostic |
| | | | assessment |
| Coronary artery disease | | | Diagnostic |
| | | | assessment |
| Shock | | | |
| Organ function | Improved | Improved | |
| Survival rate | | Increased | Increased |
| Airway mucosa function | | | |
| Surfactant secretion | Increased | | |
| Chloride secretion* | Increased | | Increased |
| Ciliary beat frequency | Increased | | |
| Mucus secretion | Increased | | |
| Water secretion | Increased | | |
| Mucociliary clearance** | | | Increased |
| Metabolism | | | |
| Gluconeogenesis | Reduced/ | | |
| | Increased | | |
| Glycogenolysis | Increased | | |
| Cancer treatment | | | |
| Weight loss | | Inhibited | Inhibited |
| Tumor growth | Inhibited | Inhibited | Inhibited |
| Chemotherapy efficacy | Increased | Increased | |
| Radiotherapy damage | Reduced | Reduced | |
| Radiotherapy efficacy | Increased | Increased | |
| Radiotherapy survival | | Increased | |
| rate | | | |

*Observed in epithelia of healthy participants and cystic fibrosis patients.
**Epithelia of cystic fibrosis patients.
AV = atrioventricular, SA = sinoatrial

### SUMMARY OF THE INVENTION

The present invention discloses for the first time a method for treating ALI and ARDS by administration of adenine nucleotides and/or adenosine and inorganic phosphate to a human host.

In particular, the present invention is concerned with a method for treating ALI and/or ARDS by administering to a human host in need thereof a member selected from the group consisting of: (a) a mixture of adenosine and/or inorganic phosphate; and (b) an adenine nucleotide wherein said adenine nucleotide containing adenosine moiety(ies) and phosphate moiety(ies) and undergoes rapid degradation to adenosine and inorganic phosphate after administration to said host.

In a preferred aspect, the present invention relates to using adenosine 5′-triphosphate (ATP) in the treatment of Acute Lung Injury (ALI) and Acute Respiratory Distress Syndrome (ARDS). ATP is administered by continuous infusion through a central venous catheter (CVC), located in the superior vena cava (SVC), or directly into the pulmonary artery through a pulmonary artery catheter (PAC) or by continuous intravenous infusion.

A number of unrelated observations and properties of ATP and adenosine as will be discussed below in conjunction with my years of experience with ATP and adenosine have lead me to now suggest the use of ATP in treating ALI and ARDS. In particular, the clinical efficacy of ATP in the treatment of ALI and ARDS is based on four well-established properties of ATP and its immediate catabolic product adenosine. (1) ATP is a selective, preferential pulmonary vasodilator. (2) Adenosine, generated effectively by infusion of ATP, is an anti-inflammatory agent. (3) ATP and adenosine exhibit anti-thrombotic, pro-fibrinolytic activities. (4) Continuous infusions of ATP were shown to produce improvements in organ and muscle functions in advanced disease patients.

### DETAILED DESCRIPTION OF THE INVENTION

ALI and ARDS are disease entities that carry a mortality rate of 35-50%. This mortality rate has only slightly decreased since the diseases were first described in the late 1960s. Sepsis, severe trauma, aspiration of gastric content, massive blood transfusion and adverse consequences of cardiothoracic surgery are the most common clinical events that place patients at risk for the development of ALI and ARDS. The entire pathophysiological spectrum of the disease is called ALI whereas ARDS refer to the more severe end of the spectrum. For ALI, the ratio of pulmonary arterial oxygen pressure (PaO2) to the fraction of inspired oxygen (FiO2) is less than or equals 300 (adjusted for barometric pressure). For ARDS, the PaO2/FiO2 ratio is defined as equal or less than 200. Despite this distinction, available data suggests that the outcomes for patients with ALI are similar to the outcomes for patients with ARDS. To date, the only known modality shown to reduce mortality is the utilization of a mechanical ventilator technique that reduces alveolar over inflation. It is widely accepted that no pharmacological intervention has been demonstrated to reduce morbidity and mortality of patients with ALI and ARDS (The ARDS Network Authors, supra).

For the initiation of the clinical protocol the criteria for ALI and ARDS are:

a. Bilateral infiltrates on chest rentgenogram.

b. Pulmonary capillary wedge pressure of less than or equal to 18 mm Hg.

c. PaO2/FiO2 ratio of equal or less than 300 regardless of the PEEP (positive end-expiratory pressure) value.

d. Acute onset of pulmonary infiltrates.

e. Presence of predisposing conditions for ALI and ARDS.

It has been found pursuant to the present invention that a host suffering from ALI and/or ARDS can be treated by being administered a member selected from the group consisting of: (a) a mixture of adenosine and/or inorganic phosphate; and (b) an adenine nucleotide wherein said adenine nucleotide containing adenosine moiety(ies) and phosphate moiety(ies) and undergoes rapid degradation to adenosine and inorganic phosphate after administration to said host.

Examples of such materials are adenosine 5′-monophosphate (AMP), adenosine 5′-diphosphate (ADP), adenosine 5′triphosphate (ATP) and mixtures of adenosine and an inorganic phosphate.

Examples of inorganic phosphates are sodium phosphate, potassium phosphate and phosphoric acid. The pH of any solution employed containing the phosphate is usually adjusted, if necessary, to about 6.0 to about 7.5 by the addition of a base such as sodium hydroxide. Usually, at least about 1 equivalent of phosphate per adenosine is employed, and preferably about 1 to about 3. In addition, pharmaceutically acceptable salts, or metal complexes, or chelates, or liposomes, or radio-nuclides of the above compounds can be used.

Preparations containing the above ingredients can be employed in a variety of conventional pharmaceutical preparations. These preparations can contain organic or inorganic material suitable for internal administration. The high solubility of AMP and/or ADP and/or ATP salts and/or adenosine and phosphate salts in isotonic aqueous solutions of sodium chloride enable administration of these agents in the form of injection or infusion of single or multiple doses. The injection or infusion can be intraperitoneal, intravenous, or intra-arterial. AMP and/or ADP and/or ATP and/or adenosine and phosphate salts are also suitable for oral, enteral, or topical application when employed with conventional organic or inorganic carrier substances.

The effective doses are in the range of about 0.1-100 mg/kg of body weight per 24 hours for oral or topical administration, and 0.01-10 mg/kg of body weight per 24 hours for injections. Intravenous, intraperitoneal, or intraarterial infusions of AMP and/or ADP and/or ATP and/or adenosine and phosphate salts in a suitable salt form is preferably administered at a rate of about 0.001-1 mg/kg of body weight per minute. The delivery of these agents can be performed using a variety of drug delivery systems including, but not limited to, pumps or liposomes.

An example of a clinical procedure in the treatment of ALI and ARDS is as follows. After determination of baseline, pre-treatment hemodynamic variables, blood gases and pH values, an ATP dose escalation procedure is initiated. ATP is provided as a sterile solution in single use vials. Each vial contains 2 grams of disodium ATP in 20 ml of Water for Injection, at pH 6.7-7.2. The concentration of ATP is 100 mg/ml. Storage of the clinical solution is at controlled refrigerated temperature (2° C.-8° C. ). Preparation of the infusion solution requires that the volume of one vial of ATP be aseptically removed using a syringe and added to a 250 ml bag of 0.5 normal saline (which has been volume corrected by removal of 20 ml of saline). The concentration of the final sterile solution for the infusion is 8 mg/ml. The stability of the final ATP solutions at room temperature is at least 96 hours.

An initial dose of 10 micrograms/kg/min of ATP is infused and after equilibration and within 30 minutes of initiation of the treatment, the original, pre-treatment set of physiological parameters is determined for this dose of ATP. The ATP dose is escalated by a stepwise procedure to 20, 30, 40 and a maximum of 50 micrograms/kg/min. Dose escalation proceeds with the same set of physiological parameters being determined, after equilibration and within 30 minutes after initiation of every new dose of ATP. The choice of the optimal dose of ATP is then made according to pre-set rules for improvement and stopping rules. Dose will be escalated as long as improvements are observed and no stopping rules apply. These two sets of rules are based on the values of mean pulmonary arterial pressure (PAP), pulmonary vascular resistance (PVR), mean systemic arterial pressure (MAB), systemic vascular resistance (SVR), cardiac index (CI), the ratio of PaO2/FiO2, mixed venous oxygen saturation, arterial oxygen tension, arterial carbon dioxide tension and pH. The optimal dose of ATP is then continuously infused for a maximum of 72 hours. An initial assessment of the treatment is made during the infusion, based on the ATP dose tolerability, lack of adverse effects, constant reduction of pre-treatment PAP with little or no decrease in MAB and improvement in blood gases levels at constant ventilation. Based on these interim values, the clinician has the following choices: to proceed with the infusion at the optimal dose, to lower the dose or to stop the infusion altogether. Ventilator management is conducted according to the ARDS Net protocol with weaning off the ventilator performed according to the adopted Ventilator Liberation Protocol (VLP) for each particular ICU (Intensive care unit). Clinical outcome measures are determined during and at the end of 30 days follow-up, after the termination of the ATP infusion. Clinical outcome measures consist of the following: 30 days mortality and the number of days with unassisted breathing (ventilator-free days) during 30 days of follow-up, ICU and hospital days, pulmonary and cardiovascular physiology values, APACHE II score, lung injury score (LIS) and organ failure measure (SOFA). The biochemical measurements that will be performed on blood samples collected at baseline and during the infusion and follow-up periods include: serum levels of lactate dehydrogenase (LDH), albumin, pre-albumine, C-reactive protein and the levels of serum enzymes indicative of hepatic function SPOT and SPGT.

My years of experience with ATP and adenosine, as discussed above have suggested to me that four random documented activities of continuous infusions of ATP contribute to the expected benefits in the treatment of ALI and ARDS. These activities are:

ATP is a selective, preferential pulmonary vasodilator. ATP and its in vivo catabolic product, adenosine, interact with receptors that regulate vascular tone. Their metabolic lability, especially due to first passage effects in the lungs, when administered by superior vena cava or pulmonary artery infusions, is the reason for the strictly pulmonary vasodilatory effects of ATP and adenosine.

Adenosine, generated in-vivo by continuous administration of ATP, is a powerful anti-inflammatory agent.

Continuous infusions of ATP produce anti-thrombotic, pro-fibrinolytic activities.

Continuous infusions of ATP produce improvements in organ and muscle function in advanced disease patients.

The rationale for utilization of continuous infusions of ATP via a central catheter (superior vena cava administration) in the treatment of ALI and ARDS is thus based on these activities of ATP acting in consort and disclosed together in this patent application. ATP administered by intravenous infusions and especially ATP administered by superior vena cava infusions, is a predominant pulmonary vasodilator. Both ATP and adenosine are powerful vasodilators acting by affecting vascular endothelial P1(A) (adenosine) and P2 (ATP) receptors. The extremely short metabolic half life of ATP and adenosine inside the vascular bed assures that their vascular effects after superior vena cava administration will be limited to the pulmonary vasculature. At doses utilized in this patent disclosure, ATP and adenosine produce immediate selective and preferential vasodilatory effects in the pulmonary circulation. These effects include significant decreases in pulmonary arterial pressure and pulmonary vascular resistance without affecting heart rate, or producing reductions in systemic arterial pressure or systemic vascular resistance (Brook et al, Use of ATP-MgCl₂ in the evaluation and treatment of children with pulmonary hypertension secondary to congenital heart defects. Circulation 1994; 90:1287-1293; Fullerton et al, 1996; Gerasimov et al. 1994). The resolution of acute and chronic pulmonary hypertension by administration of ATP or adenosine in humans (Brook et al., 1994; Fullerton et al., Adenosine for refractory pulmonary hypertension. Ann. Thorac. Surg. 1996; 62:874-877; Gerasimov et al., Biologically active substances during treatment of pulmonary hypertension with ATP infusions immediately after general anesthesia and surgery of hypervolemic congenital heart defects. Article in Russian, Anesteziol. Reanimatol. 1994; 3:14-17.; Gaba et al. Comparison of pulmonary and systematic effects of adenosine triphosphate in chronic obstructive pulmonary diseases-ATP: a pulmonary controlled vasoregulator? Eur. Respir. J. 1990; 3:450-455; Colson et al., Effets vasculaires del'adenosine-triphosphate. Ann. Fr. Anesth. Reanim. 1991; 10:251-254), and in experimental animals (Kaapa et al., 1997; Paidas et al., Adenosine triphosphate: A potential therapy for hypoxic pulmonary hypertension. J Pediatr Surgery 1988; 23(12):1154-60; Fineman et al., 1990; Konduri, 1994) is documented. ATP release by red blood cells is now being proposed as the physiological mechanism for the local control of the pulmonary circulation (Sprague et al., 1996). ATP acts in regulating the pulmonary vasculature by three independent mechanisms, nitric oxide (NO)-dependent, adenosine and non-NO dependent and non-adenosine and non-NO-derived mechanisms. Recently, the failure of red blood cells to release ATP in patients with primary pulmonary hypertension (PPH) was demonstrated. The impaired ability of red blood cells to release ATP in the pulmonary vascular bed was proposed as the major pathogenic factor accounting for the heretofore unknown etiology of PPH (Sprague et al., Impaired release of ATP from red blood cells of humans with primary pulmonary hypertension. Exp Biol Med 2001; 226:434-439).

Adenosine, generated in vivo by the continuous administration of ATP is a powerful anti-inflammatory agent. The modulation of neutrophil function by adenosine is currently being utilized in experimental treatment of inflammatory conditions as rheumatoid arthritis and reperfusion injury (Cronstein Adenosine regulation of neutrophil function and inhibition of inflammation via adenosine receptors. In “Purinergic Approaches in Experimental Therapeutics”, Jacobson K A and Jarvis M F, Eds., Wiley-Liss, Inc. 1997; Linden, Molecular approach to adenosine receptors: receptor-mediated mechanisms of tissue pritection. Annu Rev Pharmacol Toxicol 2001; 41:775-787).

Both adenosine and ATP have been utilized in the treatment of the inflammatory and hemodynamic consequences of septic shock in experimental animal models (Rosengren et al Purinergic modulation of septic shock and systemic inflammation response syndrome. In “Purinergic Approaches in Experimental Therapeutics”, Jacobson K A and Jarvis M F, Eds., Wiley-Liss, Inc., 1997 and Wang et al., Salutary effects of ATP-MgCl2 on the depressed endothelium-dependent relaxation during hyperdynamic sepsis. Crit Care Med 1999; 27:959-964). The anti-inflammatory activities of adenosine have been recently proposed to account for the mechanism of action of several established anti-inflammatory drugs. These drugs were demonstrated to produce release of cellular ATP in vivo, followed by the rapid degradation of the ATP to adenosine, which mediated the observed anti-inflammatory activities (Morabito et al., 1998; Cronstein et al., Site of action for future therapy: an adenosine-dependent mechanism by which aspirin retains its antiinflammatory activity in cyclooxygenase-2 and NFkappaB knockout mice. Osteoarthritis Cartilage 1999; 7:361-363).

ATP and adenosine are established inhibitors of platelet aggregation, acting not only by binding to their own receptors but also antagonizing the binding of adenosine di-phosphate (ADP) in its promotion of platelet aggregation. Recently, additional adenosine-dependent mechanisms of induction of coronary thrombolysis were described (Przyklenk et al. Brief antecedent ischemia enhances recombinant tissue plasminogen activator-induced coronary thrombolysis by adenosine-mediated mechanism. Circulation 2000; 102:88-95). ATP infusions in humans have been shown to induce a profound acute release of tissue-type plasminogen activator (tPA). The release of ATP in vivo in ischemic tissue has been propose to induce a significant activation of the endogenous fibrinolytic system (Hrafnkelsdottir et al., Extracellular nucleotides ATP and UTP induce a marked acute release of tissue-type plasminogen activator in vivo in man. Thromb Haemost 2001, 85:875-881.). The anti-thrombotic and pro-fibrinolytic properties of both ATP and adenosine provided a rationale for their use in experimental treatment of acute myocardial infarction in human clinical trials (Kitakaze et al., Adenosine and cardioprotection of the diseased heart. Jpn Circ J 1999; 63:231-243; Nakayama et al., Intracoronary administration of ATP combined with direct PTCA reduces the size of myocardial infarction?: Cooperative Osaka adenosine trial for acute myocardial infarction (COAT). J. Amer. College Cardiol. 1999; 33: 376, Abstract).

In addition to its pronounced activities in resolving pulmonary hypertension, in reducing inflammatory reactions and in promoting anti-thrombotic and pro-fibrinolytic activities, ATP has been documented to improve organ function in multiple organ failure (MOF) patients (Hirasawa et al., 1989). ATP was also shown to positively affect weight, muscle function and quality of life parameters in cachectic, advanced non-small cell lung cancer patients (Agteresch et al., 2000, supra). Multiorgan dysfunction is a major contributor to morbidity and mortality in ALI and ARDS patients and recovery from ALI and ARDS is dependent on adequate support of all vital organ systems (Kollef et al, 1995, supra). Respiratory muscle strength and its direct relationship to muscle ATP levels have been a recent therapeutic target in the treatment of cachectic chronic obstructive pulmonary disease (COPD) patients (Marchesani et al., Effect of intravenous fructose 1,6-diphosphate administration in malnourished chronic obstructive pulmonary disease patients with chronic respiratory failure. Respiration 2000; 67:177-182).

Examples of activities of ATP, which by acting in consort contribute to the decrease in morbidity and mortality in ALI and ARDS patients are:

A. Vascular Tone.

Both P1 (adenosine) and P2 (ATP) receptors are present on vascular endothelial and perivascular cells and both play a major role in the regulation of vascular tone. Two subtypes of ATP receptors have been characterized: P2X and P2Y receptors. P2X receptors are located on smooth muscle cells and upon activation induce vasoconstriction. P2Y and P1 receptors are located on vascular endothelial cells and upon activation mediate a vasodilatory response. It is the endothelium-dependent vasodilation that accounts for a variety of activities of extracellular ATP inside the vascular bed.

When administered intravenously, low doses of ATP and adenosine have predominantly pulmonary rather than systemic vasodilatory effects. The reason being their rapid metabolism during first passage through the lungs. In the pulmonary vascular bed the predominant types of purine receptors are the P2Y and P1 receptors (Reviewed by Agteresch et al., 1999, supra). Binding of ATP to P2Y receptors activates NO synthetase which in turn induces a second messenger system, resulting in smooth muscle relaxation. ATP also activates, through P2Y receptors, prostacyclin synthesis, which is a vasodilatory agent as well as producing a non-prostacyclin, non-NO vasodilation (Rongen et al., Characterization of ATP-induced vasodilation in the human forearm vascular bed. Circulation 1994; 90:1891-1898).

Extracellular adenosine activation of P1 receptors on pulmonary vascular endothelial cells results in significant vasodilation through mechanisms that involve increases in cellular adenyl cyclase activity by a second messenger system.

Thus, administration of ATP or adenosine directly into the pulmonary artery or into the superior vena cava in humans produces selective predominant pulmonary vasodilation through a first passage effect. The effects are in reducing pulmonary arterial pressure and pulmonary vascular resistance without any direct effect on systemic arterial pressure or systemic vascular resistance (Brook et al., 1994, supra; Fullerton et al., 1996, supra; Gerasimov et al., 1994, supra). This outcome is due to the metabolic lability of ATP and adenosine in the pulmonary vasculature, resulting in their degradation during first passage and therefore no effects on the systemic vasculature.

The first passage effects of ATP versus those of adenosine have produced differences between these two agents in the regulation of vascular tone. ATP, during first passage, as is expected in the pulmonary vasculature after superior vena cava infusion, acts by a variety of mechanisms, including through its catabolic product, adenosine. Thus ATP, during first passage, has been shown previously to possess more pronounced and longer lasting effects than the comparable effects achieved by first passage adenosine (Rongen et al., supra; Kato et al., Adenosine 5′ triphosphate induces dilation of human coronary microvessels in vivo. Internal Medicine 1999; 38:324-329).

It is important to note that ATP, but not adenosine induced a marked release of tissue type plasminogen activator (tPA) in humans (Hrafnkelsdottir et al., supra), an effect expected to beneficially affect its activities in the treatment of ALI and ARDS. ALI and ARDS accompanied pulmonary hypertension is thought to be due to activation and release of vasoactive mediators, including arachidonic acid metabolites, followed by diffuse pulmonary microvascular thrombosis (The ARDS Network Authors, supra). ATP, as well as adenosine, has been in clinical trials for acute myocardial infarction (Nakayama et al., supra).

B. Pulmonary Hypertension.

Acute pulmonary hypertension is a serious clinical problem after thoracic surgery in patients with chronic obstructive pulmonary disease (COPD) and in cardiac surgery for repair of congenital heart defects. Vasodilators have often been unsuccessful, since they act simultaneously on the pulmonary and systemic vasculature with predominant systemic effects (Rubin, 1987). Low dose intravenous infusions of adenosine and ATP however, have produced predominant, selective pulmonary vasodilatory effects in humans (Colson et al., 1991; Gaba et al., 1986; Gaba and Prefaut, 1990; Shiode et al., 1998; Fullerton et al., 1996).

In newborn lambs, often utilized as experimental animals for pulmonary hypertension, low doses of intravenous ATP were effective in resolving pulmonary hypertension (Paidas et al., 1988; Fineman et al., 1990; Konduri et al., 1992). Studies of experimental animals and human clinical trials have demonstrated that intravenous infusions of up to 100 micrograms/kg/min of ATP, produce predominant decreases in pulmonary arterial pressure and pulmonary vascular resistance. Systemic arterial pressure and systemic vascular resistance are affected only by higher rates of ATP infusions (Gaba and Prefaut, 1990; Fineman et al., 1990; Paidas et al 1989; Kappa et al., 1997;Shiode et al., 1998).

In humans, studies of healthy individuals (Utterback et al., 1994;Reid et al., 1990), COPD patients (Gaba and Prefaut, 1990; Gaba et al., 1986), children with pulmonary hypertension after surgical repair of congenital heart defects (Brook et al., 1994) or patients after surgical repair of hypervolemic congenital heart valve defects (Gerasimov et al., 1994), demonstrated the efficacy of ATP in significantly decreasing pulmonary arterial pressure and pulmonary vascular resistance. In seven children with life-threatening pulmonary hypertensive crises after surgical repair of congenital heart defects, ATP was effective in completely resolving the hypertension crises and restoring hemodynamic stability in 4 children. These children survived and no rebound pulmonary hypertension was noted. The ATP in these cases was infused directly into the pulmonary artery for periods of 30 minutes to 30 hours (median of 6 hours) (Brook et al., 1994).

Fullerton et al., (1996) reported two cases of patients with acute life-threatening pulmonary hypertension after cardiac and thoracic surgeries. In both cases the patients were completely refractory to several vasodilators including continuous nitric oxide inhalation. In both instances, adenosine infusion into the superior vena cava effectively lowered pulmonary arterial pressure and increased systemic arterial pressure and cardiac index when all available standard treatment measures have failed. The clinical state of shock was reversed and the patients were successfully weaned from mechanical ventilation.

C. Inflammation.

Experimental data, based mostly on pre-clinical experimental animal data, document the mechanisms of the anti-inflammatory activities of adenosine. Three recent reviews discuss the adenosine-mediated inhibitory effects against inflammatory processes in septic shock and systemic inflammation response syndrome (SIRS) (Rosengren and Firestein, 1997; Cronstein, 1997; Linden, 2001). The adenosine is generated either endogenously, as an ingredient of the host defense mechanisms under conditions of metabolic stress or ischemia, or is used exogenously as a pharmacological agent. The endogenously produced adenosine is generated from ATP by the activities of catabolic enzymes. It has been demonstrated recently that the anti-inflammatory effects of methotrexate and sulfasalazine are mediated by an in vivo adenosine release mechanism (Morabito et al., 1998). The active extracellular adenosine however, is generated by the degradation of ATP, which is in turn released from target cells affected by these drugs. The inhibitory effects of adenosine were established against the various processes involved in inflammation (Reviewed by Agteresch et al., 1999). These include inhibition of the major aspects of neutrophil function, inhibition of the synthesis of inflammatory cytokines and significant reduction of endothelial permeability. It is the activation of adenosine A2alpha receptors that produces the anti-inflammatory cell activities. Activation of A1 and possibly A3 adenosine receptors is responsible for the tissue protective activities of adenosine, by preconditioning mechanisms involving protein kinase C and KATP channels (Reviewed by Linden, 2001).

The cardiovascular effects of ATP and adenosine are significant in improving blood flow after ischemia. In pre-clinical animal models of shock, intravenous ATP enhances renal and hepatic microcirculation, portal and total hepatic blood flow and cardiac output (Reviewed by Wang et al., 1999).

D. Shock .

An important clinical feature of shock is the inadequate circulation with diminished perfusion of tissues, resulting in hypoxia and internal injury. The resuscitation period after shock is commonly associated with tissue injury and impairment of organ function mostly due to reperfusion injury. Reperfusion injury (RI), is the result of inflammatory cell adhesion to ischemic tissue and the release of a barrage of injurious oxygen metabolites and proteolytic enzymes from the inflammatory leukocytes (Cronstein, 1997). Several studies of ATP administration in hemorrhagic shock-trauma animal models have demonstrated significant positive effects on survival (Reviewed by Agteresch et al., 1999; Wang et al., 1999). Other pre-clinical studies showed that ATP and adenosine infusions have protective effects on tissue injury during reperfusion after a preceding period of ischemia. Functions of rat kidney, rat lung, dog heart, rabbit lung and rat gut, were improved under these treatments. Intramuscular ATP was proven effective in a rat burn model in protecting the intestinal mucosal structure and function (Reviewed by Agteresch et al., 1999).

Clinically, ATP administration was shown to be beneficial to several metabolic functions in multiple organ failure patients (Hirasawa et al., 1989). No survival data could be established since the study was a single arm study. However, metabolic changes that were associated with survival versus non-survival, were established to be associated also with ATP administration in this patient population.

E. Advanced Cancer.

ATP is being developed as a treatment for advanced refractory cancers, based on issued U.S. patents to Rapaport, and several clinical trials have provided extensive experience in continuous intravenous infusions of ATP in advanced disease patients. Published trials of ATP administration in advanced cancer patients included continuous intravenous infusions of 20-100 micrograms/kg/min of ATP for 96 hours every 4 weeks or for 30 hours every two weeks. In the initial Phase I/II clinical trial, ATP was administered as a continuous intravenous infusion for 96 hr, once every 4 weeks, at rates of 50, 75 or 100 microgram/kg/min (Haskell et al. 1996). The trial included 14 patients with advanced cancer, eight of whom suffered from stage IIIB/IV non-small cell lung cancer. Most of the patients were chemotherapy-naive. One patient received one infusion; 4 received 2 infusions; 6 received 3 infusions; 1 received 4 infusions; and 2 received 6 infusions. The dose-limiting toxicity seen in this study was a cardiopulmonary reaction characterized by tightness of the chest and what has been described as an urge to take a deep breath that resolved immediately after discontinuing the ATP infusion. This reaction was seen in all three patients (100%) infused at 100 microgram/kg/min; in 3 of 6 (50%) patients infused at 75 microgram/kg/min; and, in 4 of 11 patients (36%) who received 50 microgram/kg/min. In some cases, this reaction was accompanied by electrocardiographic changes suggestive of myocardial ischemia. Less frequent or less prominent adverse effects that may, or may not, have been related to ATP treatment were injection site reactions (“local reactions,” pain and phlebitis), hypoxia, hypotension, ECG abnormalities, nausea and/or emesis, abdominal pain, dizziness, headache, anxiety, back or neck pain, anemia, and leukopenia. With respect to the pharmacokinetic properties of ATP, Haskell et al. (1996) measured the whole blood concentrations of ATP of 18 subjects before and at 24, 48, 72, and 96 hr during and after infusions at 50, 75, or 100 microgram/kg/min. The ATP concentrations were found to vary widely, but, in general, they increased 30%-40% after 4 hr and the highest blood concentrations were seen at 24 hr of infusion. These concentrations averaged 63%, 67%, and 113% respectively higher than the pretreatment values. The blood concentrations of ATP were relatively constant or slightly declined during the interval between 24 and 96 hr of the infusion. Little data are available concerning the decay of ATP concentrations post-infusion. These authors concluded that prolonged infusions of ATP are feasible with acceptable toxicity and that 50 microgram/kg/min is both the maximum tolerated dose and the most appropriate dose rate for subsequent Phase II testing of 96-hr infusions of ATP in patients with advanced non-small cell lung cancer. Patients suffering from other cancers could easily tolerate the highest dose of 100 microgram/kg/min

The second trial conducted by Mendoza et al. (1996) was a Phase II multicenter study of 15 chemotherapy-naïve, stage IIIB/IV, advanced non-small cell lung cancer patients who were infused at 50 or 65 microgram/kg/min for 96 hr, once every 4 weeks. Two patients received 1 infusion; 8 received 2 infusions; 4 received 4 infusions; and 1 received 7 infusions. A proportion of patients in this study experienced a variety of adverse effects, including chest pain, coughing, anxiety, injection site pain, chest tightness, an urge to take a deep breath, headache, insomnia, and hot flashes. Almost one-half of the patients exhibited abnormal electrocardiograms. In some patients, there were minor reductions in hematocrit, hemoglobin, total protein, albumin, sodium, and calcium and minor increases in serum glucose. No significant hematologic, renal, hepatic, or gastrointestinal toxicity was noted. Although no significant tumor shrinking was observed, the majority of patients exhibited stable disease after treatment with ATP. In addition, beneficial effects were seen on weight gain, patient performance status and significantly on the overall survival of patients with non-small cell lung cancer as compared with historical data.

The third trial was reported by Agteresch et al., (2000). It was a randomized, open label Phase III study of 53 previously treated, refractory, stage IIIB/IV, advanced non-small cell lung cancer patients who failed previous chemotherapy and/or radiation therapy. The patients were randomized into two groups. One group of 26 patients received best supportive care and infusions of ATP while the other group of 27 patients received only best supportive care. The ATP-treated patients received ten 30-hr infusions, the first seven at 2-week intervals and the last three at 4-week intervals. In each case, the infusion was started at 20 microgram/kg/min and increased every 30 min by 10 microgram/kg/min until adverse effects developed or a maximum dose of 75 microgram/kg/min was reached. If adverse effects developed, the dose was reduced stepwise until the adverse effects disappeared. Eleven patients received 1-3 infusions; five received 4-6 infusions; and 12 received 7-10 infusions.

The adverse experiences seen during the phase III trial were generally mild and consisted of chest discomfort, urge to take a deep breath, flushing, nausea, lightheadedness, headache, sweating, anxiety, and palpitations. In patients with chest discomfort, electrocardiograms did not exhibit changes suggestive of myocardial ischemia. All side effects resolved within minutes of lowering the rate of ATP infusion with continuation of the infusion at a lower rate. In this study, ATP administration was associated with beneficial effects on body weight and voluntary muscle strength and with improvements in Quality of Life domains. Thus, the authors concluded that ATP shows promise as an agent for the palliation of cancer cachexia in advanced, refractory cancer patients (Agteresch et al., 2000).

Pharmacology of ATP.

Cardiovascular Effects.

A number of studies that have described the effects of continuous intravenous infusions of ATP on the cardiovascular system in anesthetized animals are summarized in Table 2.

TABLE 2
Effects of Intravenous Infusions of ATP in Experimental Animals

| | ATP Infusion Rate | |
|---|---|---|
| Parameters | ≦100 μg/kg/min | 500-1000 μg/kg/min |
| Heart Rate | No Change | Increase |
| Systemic Arterial Pressure | No Change | Decrease |
| Pulmonary Arterial Pressure | Decrease | Decrease |
| Cardiac Output | Small Increase | Increase |
| Systemic Vascular | Small Decrease | Decrease |
| Resistance | | |
| Pulmonary Vascular | Decrease | Decrease |
| Resistance | | |

Data from Baba et al. 1989 (rats); Fukunaga et al. 1982 (rabbits); Newberg et al. 1985 (dogs); Paidas et al. 1988 (piglets); Van Aken 1984a (dogs); Van Aken et al. 1984b (baboons).

At relatively low rates of infusion, generally at 100 microgram/kg/min or less, intravenous infusions of ATP produced changes primarily in pulmonary hemodynamics, with little, if any, changes in the systemic circulation. These infusions decreased pulmonary artery pressure and pulmonary vascular resistance, but did not alter systemic arterial pressure although there was a small decrease of systemic vascular resistance that was offset by a small increase in cardiac output.

In studies in which ATP was infused at higher rates, treatment led to more distinct effects on both the pulmonary and the peripheral circulation. These infusions produced decreases in both pulmonary and peripheral arterial pressure and in both pulmonary and peripheral vascular resistance (Table 2).

Physical, Chemical, and Pharmaceutical Properties (Drug Product)

ATP is provided as sterile liquid in 20 ml vials. Each vial contains 2.0 grams of ATP in 20 ml of water for injection USP (100 mg ATP/ml), pH adjusted with sodium hydroxide. The product must be stored at controlled refrigerated temperature (2° C.-6° C.). At pH 6.7-7.2, the sodium salt in aqueous solution is stable.

Structure.

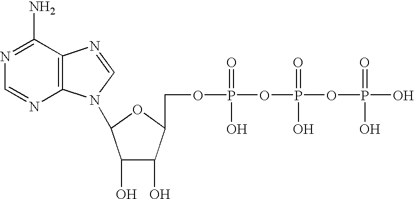

Drug Distribution, Metabolism, and Elimination.

Distribution.

Not surprisingly, since it is a major source of cellular energy, ATP is widely distributed, being found in every cell.

Metabolism.

It is well known that ATP is metabolized in man and the Dalmatian dog via the following series of metabolites: (1) adenosine 5′ diphosphate (ADP), (2) adenosine monophosphate (AMP), (3) adenosine, (4) inosine, (5) hypoxanthine, (6) xanthine, and (7) uric acid. In other mammals, uric acid is oxidized to allantoin. Many of these intermediates are recycled back into selected biochemical pathways in most organs but the kidney, each to a variable extent dependent on the species, excretes uric acid and allantoin.

Elimination.

As mentioned above, ATP is metabolized in several steps to uric acid, and in some species to allantoin. These metabolites are then excreted by the kidney in a species-dependent manner.

Toxicology.

Acute Toxicity.

There do not appear to be any published studies that examined the acute toxicity of ATP in unanesthetized animals.

Repeated-Dose Toxicity.

There do not appear to be any published studies that examined the toxicity of ATP associated with repeated dosing of unanesthetized animals.

Mutagenicity, Reproductive Toxicity, and Carcinogenicity.

Studies have not been performed to evaluate the mutagenicity, reproductive toxicity, or carcinogenicity of ATP in animals. Adenosine, a metabolite of ATP in animals and man (see Agteresch et al. 1999), has tested negative for genotoxic potential in the Salmonella (Ames Test) and the Mammalian Microsome Assay although it is known to produce a variety of chromosomal alternatives in cell cultures (Physicians' Desk Reference, 1999). In rats and mice, adenosine administered intraperitoneally once a day for 5 days at 50, 100, and 150 mg/kg caused decreased spermatogenesis and increased numbers of abnormal sperm, reflecting perhaps its ability to produce chromosomal damage (Physicians' Desk Reference, 1999).

Pregnancy Category C.

Since the ability of ATP to interfere with reproduction has not been studied in either animals or human subjects, it should not be administered to pregnant women or to women of reproductive potential.

Safety and Efficacy.

Although rare, the following serious and potentially life-threatening complications have been associated with intravenous infusions of adenosine (a known metabolite of ATP) when adenosine infusion was at a rate higher than the planned rate of infusion of ATP: severe bronchospasm (0.03%), nonfatal myocardial infarction (0.02%), severe hypotension (0.45%), and severe bradycardia (0.04%). Such complications should be managed as clinically indicated and with recording of the event on the case report form.

Having thus described my invention, what I claim as new and useful and desire to secure by Letters Patent is:

1. A method for preventing and/treating acute lung injury (ALI) and/or acute respiratory distress syndrome (ARDS) by administering to a human patient in need thereof a member selected from the group consisting of: (a) a mixture of adenosine and inorganic phosphate; (b) an adenine nucleotide wherein said adenine nucleotide containing adenosine moiety(ies) and phosphate moiety(ies) and undergoes rapid degradation to adenosine and inorganic phosphate after administration to said patient; and mixtures thereof.

2. The method of claim 1 wherein acute lung injury (ALI) is prevented and/or treated in a human patient by administering to said patient adenosine and/or adenosine 5′-monophosphate and/or adenosine 5′-triphosphate.

3. The method of claim 1 wherein acute respiratory distress syndrome (ARDS) is prevented and/or treated in a human patient by administering to said patient adenosine and/or adenosine 5′-monophosphate and/or adenosine 5′-triphosphate.

4. The method of claim 1 wherein adenosine or adenine nucleotide are administered to a human patient as pharmaceutically acceptable salts thereof, or chelates thereof, or metal complexas thereof, or liposomes thereof.

5. The method of claim 1 wherein adenosine is administered to said patient.

6. The method of claim 1 wherein adenosine 5′-monophosphate is administered to said patient.

7. The method of claim 1 wherein adenosine 5′-triphosphate is administered to said patient.

8. The method of claim 1 wherein the amount of adenine nucleotide is about 1-500 micrograms/kg of body weight per minute and said administering is by infusion.

9. The method of claim 1 wherein the amount of adenosine is about 1-500 micrograms/kg of body weight per minute and said administering is by infusion.

10. The method of claim 1 wherein the amount of adenine nucleotide is about 10-50 micrograms/kg of body weight per minute and said administering is by infusion.

11. The method of claim 1 wherein the amount of adenosine is about 10-50 micrograms/kg of body weight per minute and said administering is by infusion.

12. The method of claim 1 wherein the amount of adenine nucleotide is about 0.01-10 milligrams/kg of body weight per 24 hours and administering is by injection.

13. The method of claim 1 wherein the amount of adenosine is about 0.01-10 milligrams/kg of body weight per 24 hours and administering is by injection.

14. The method of claim 1 wherein the amount of adenine nucleotide is about 0.1-100 milligrams/kg of body weight per 24 hours and administering is oral.

15. The method of claim 1 wherein the amount of adenosine is about 0.1-100 milligrams/kg of body weight per 24 hours and administering is oral.

16. A process for preventing and/or treating acute lung injury (ALI) and/or acute respiratory distress syndrome (ARDS) in a human patient in need thereof by increasing organ, blood and blood plasma levels of adenosine 5′-triphosphate in said patient.

17. A process for preventing and/or treating acute lung injury (ALI) in a human patient in need thereof by increasing organ, blood and blood plasma levels of adenosine 5′-triphosphate in said patient.

18. A process for preventing and/or treating acute respiratory distress syndrome (ARDS) in a human patient in need thereof by increasing organ, blood and blood plasma levels of adenosine 5′-triphosphate in said patient.

19. The process of claim 16 wherein said treating a human patient is with at least one agent selected from the group of adenosine and/or adenosine 5′-monophosphate and/or adenosine 5′-triphosphate.

20. The process of claim 16 wherein said treating a human patient is with a dose of about 1-500 micrograms/kg of body weight per minute and active agent is administered by infusion.

21. The process of claim 16 wherein said treating a human patient is with a dose of about 0.01-10 milligrams/kg of body weight per 24 hours and active agent is administered by injection.

22. The process of claim 16 wherein said treating a human patient is with a dose of about 0.1-100 milligrams/kg of body weight per 24 hours and active agent is administered orally.
